(19) United States
(12) Patent Application Publication
(10) Pub. No.: US 2008/0215072 A1
(43) Pub. Date: Sep. 4, 2008

(54) METHODS AND APPARATUS FOR UTILIZATION OF BARBED SUTURES IN HUMAN TISSUE INCLUDING A METHOD FOR ELIMINATING OR IMPROVING BLOOD FLOW IN VEINS

(76) Inventor: Graham Kelly, Old Mission, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/017,191

(22) Filed: Jan. 21, 2008

Related U.S. Application Data
(60) Provisional application No. 60/889,951, filed on Feb. 15, 2007.

(51) Int. Cl. A61B 17/12

(52) U.S. Cl. 606/151; 128/898

(57) ABSTRACT

A method and apparatus for eliminating or improving blood flow within incompetent veins to correct venous insufficiency using a barbed bidirectional suture with predetermined breaking point. Said suture may also be utilized for other tissue applications. A two-way barb suture is placed in an insertion device comprised of a tubular body with or without a pointed distal tip. The inserting device and one-way or two-way suture are placed in a position to effectively close off the vein or leave a device within the vein. The insertion device is then withdrawn leaving the suture in place. This barbed suture with predetermined breaking point is used by the method of the present invention to prevent reflux, and as a method to join body tissue, attach dissimilar body tissues, attach devices to body tissues, and alter the position of body tissues by remote percutaneous access with the assistance of ultrasound or fluoroscopy and with the assistance of endoscopic devices.

### BACKGROUND OF THE INVENTION

This invention relates generally to the human venous system, specifically to the modification or elimination of poorly or nonfunctioning veins within this system and to methods and apparatus suitable for improving blood flow in veins.

Over 25 million Americans have varicose veins. It has been estimated that over 4.6 million work days are lost each year due to the effects of venous disease with costs totaling in the billions of dollars. The direct government cost for treating advanced venous disease exceeds one billion dollars per year.

Veins rely on one-way valves that permit blood flow only in one direction. With loss of vein valve function, blood does not flow properly and only repair of the valves or elimination of the malfunctioning vein will normalize circulation in the venous system. To understand the function of the proposed venous modification devices, it is necessary to review basic venous anatomy.

The human venous system is comprised of the superficial, perforator, and deep systems. Blood flow direction is from the superficial system, which drains superficial structures such as the skin and subcutaneous tissues, through the perforator veins to the deep venous system. From there, blood is returned to the heart where it is pumped through the lungs to be re-oxygenated. Blood is then returned to the heart to be pumped back through the tissues where oxygen is utilized. The de-oxygenated blood is then returned to the venous system, and the cycle is then repeated. Perforator veins are crucial connections allowing blood flow from the superficial to the deep venous systems.

Blood flow in the venous system can be compared to circulation of water in the root system of a tree. There are many small roots leading to progressively larger roots and eventually leading to a main trunk root. Cutting or injuring many small roots would typically have minimal impact on the tree, but damage of the main trunk root can cause major problems or even loss of the tree. In humans, the venous system is similar, in that the superficial veins are plentiful much like the small roots of a tree, and smaller veins flow into progressively larger veins. Injury to the superficial veins can often be compensated for because of the large redundant capacity of the superficial venous system. The deep system is much like the main trunk root of a tree, and loss of parts of this system can be serious or even fatal as there is very little redundant capacity or alternative pathways for blood flow.

In the venous system, one-way valves are utilized to force blood flow in one direction back to the heart and prevent blood from pooling. With the assistance of constricting muscles that compress veins and the blood within them, circulating blood flows toward the heart and against the force of gravity. This is normally a very efficient system. However, poor or absent valve function, known as venous incompetence, either from acquired or genetic weaknesses or from injury can allow blood to pool in the legs. The end results can be the development of many symptoms including pain, itching, and swelling. Varicose veins may also develop, and in severe cases open wounds or venous ulcers can occur.

Conventional treatment utilizes graduated compression hosiery and, in effect, pushes blood out of the leg towards the heart. These are by necessity tight and are typically hot and uncomfortable for most who wear them. Surgical techniques can remove some poorly functioning veins but the treatment is invasive and fraught with serious potential complications and long patient recovery periods.

Newer techniques have been developed utilizing laser or radiofrequency-admitting fibers that are inserted into veins to allow for heat-related ablation of the vessels internally. These methods work well for some superficial veins but are not appropriate for deep vein treatment. Utilizing these methods for perforator veins, while possible, carries increased risk of damaging the deep venous system due to its close proximity. Sclerotherapy techniques allow for injection of veins with a sclerosant that also causes vein destruction. This technique is also inappropriate for the deep venous system, and carries significant risk when utilized in the perforator system, again due to the close proximity and tie potential risk of damaging that system.

In summary, venous insufficiency treatment utilizing heat from lasers or radiofrequency fibers can cause collateral damage to surrounding structures including the deep venous system. Sclerotherapy ablation has the well-documented risk of sclerosant migration into surrounding veins including the deep venous system. These problems limit the usefulness of all of those currently utilized techniques.

One of the most common problems seen in patients with serious venous disease or venous ulcers is incompetent perforator veins. Loss of valve function in these veins allows backward flow of blood directly from the deep system into the superficial system, the reverse of normal circulation. The resultant high pressures in the skin and surrounding structures can cause destructive tissue changes. The skin will typically darken and turn hard and fibrotic. Ulcers will often occur at this stage. Stopping reverse blood flow through these incompetent perforator veins can have a very rapid and beneficial effect on patients with vein disease. Pain is reduced, normal skin color may return, and tissues typically soften and ulcers heal when this reverse blood flow is eliminated.

However, treatment of incompetent veins, particularly perforators, has been problematic. Open surgery requires one or more large incisions, often through fibrotic skin and tissue, and complications such as poor incision healing can occur. Endoscopic perforator surgery utilizes smaller incisions to place an endoscopic ligation tool under the skin. Perforator veins are then ligated by endoscope visualization, but there is no way to differentiate incompetent from competent (normal) perforator veins so both normal and nonfunctioning perforator veins are ligated with this procedure. This indiscriminate ligation can cause an adverse impact on normal venous blood flow. As previously stated, laser and radiofrequency devices utilizing heat have the potential to cause collateral damage, and sclerotherapy can cause damage to nearby untreated veins inadvertently.

The ideal method to eliminate incompetent veins or the blood flow within them would, therefore, utilize percutaneous access for minimal invasiveness and skin disruption, would not utilize heat or chemicals to ablate the veins, thus minimizing the potential for collateral damage, and would be selective in that only incompetent veins would be targeted. Ligation or tying off veins, specifically perforator veins, to eliminate blood flow is the present standard of care. This is currently performed by making an open incision, encircling the vein with suture material and tying a firm knot or a smaller incision is utilized for the placement of endoscopic devices, winch can also accomplish this task. Unlike these current methods, the proposed method and apparatus of the present invention would significantly reduce the risk of collateral damage and can be performed by remote percutaneous access. It would also allow for treatment of veins by extravascular means, as the very small and tortuous perforator system is often inaccessible by an intravascular route.

A percutaneous method and apparatus for wound closure utilizing a barbed form of suture could be modified for such a purpose and would allow for ligation of incompetent veins without the need for open incisions, hand-tied sutures, or indiscriminate ligations by an endoscopic approach. This suture is demonstrated in U.S. Pat. No. 7,056,331, and has been FDA approved as a safe and efficacious medical apparatus. U.S. Pat. No. 7,056,331 discloses the utilization of bidirectional barbed suture material for closing wounds, joining a foreign element to tissue, and altering the position of tissue. However, utilization of the suture as a method and apparatus to close vessels with the purpose of eliminating reflux is neither claimed nor disclosed. Furthermore, this patent suggests the use of bidirectional suture for valve placement surgery only through endoscopic means and not by the remote placement by percutaneous access.

Previous attempts at selective incompetent perforator vein elimination have met with limited success at significant risk to patients. Furthermore, the scope of U.S. Pat. No. 7,056,331 does not consider integration of a one-way valve into the suture or the use of this suture to place a foreign element in the intravascular or potential space between vein walls. Also, the barbed suture of the '331 patent cannot be remotely placed by percutaneous means as there is no ability to remotely sever a length of suture in place. U.S. Pat. No. 7,056,331 utilizes an intravascular device for placing foreign elements into or through the wall of a luminal anatomical structure. The external closing or ligation of the vessel or attachment of intraluminal space foreign elements by utilization of a device placed by an extravascular approach is not disclosed.

U.S. Pat. No. 5,342,376 covers use of an inserting device for a barbed tissue connector. Said device is utilized for wound closure, and a primary advantage stated is its ability to preserve blood flow rather than prevent it. U.S. Pat. No. 5,931,855 utilizes a barbed suture with needle attachment, and is used for wound closure and cosmetic surgery only.

Accordingly, there has been a long felt and unsolved need for such a new device, which eliminates the disadvantages, noted above.

There is a need for a barbed suture that provides for vein ligation or the placement of valves to treat venous insufficiency by remote percutaneous access. Current methods would require open surgical access, or if barbed sutures were to be utilized, placement of longer than required sutures reaching to near the skin's surface. Both of these methods would subject patient to unwarranted risks.

### SUMMARY OF THE INVENTION

Disclosed herein is a novel bidirectional suture using either barbs created by cutting part way into the suture or structural barbs built into the suture or placed on it. The bidirectional suture of the present invention may be directly positioned by remote percutaneous placement of the barbed suture in a rigid form or through an inserting device to either: (1) close veins from an extravascular approach; (2) place integral one-way valves within the lumen of the vessel and for the purpose of creating one-way blood flow; or (3) anchor intravascular placed devices within the lumen of the vessels from an extravascular approach.

The present invention achieves several advantages. It provides treatment for venous reflux disease by providing a means to close malfunctioning veins and/or to place one-way valves within these veins. The invention allows percutaneous access to the vein and obviates the need for open incisions, lasers, radiofrequency devices, general anesthetics, or injection of sclerosant, all of which carry significant risks to patients. The method and apparatus of the present invention also allows for precise targeting of incompetent veins and avoids the indiscriminate ligation that occurs with endoscopic perforator ligation. The invention can be utilized with complete comfort in minutes under a local anesthetic, and the patient is immediately ambulatory. The anticipated result would include: (1) markedly reduced symptoms of venous disease; (2) normalization of venous blood flow patterns; and (3) healing of ulcers.

Still further advantages will become apparent from the study of the following description and accompanying drawings.

### BRIEF DESCRIPTION OF THE DRAWINGS

The following detailed description may best be understood with reference to the following detailed description in which:

FIG. 1 is a cross-sectional view showing bidirectional suture according to the present invention in position in the subcutaneous tissue before insertion into a vein;

FIG. 2 shows the suture of FIG. 1 fully inserted and prior to withdrawal;

FIG. 3 shows the same sutures in the process of being withdrawn;

FIG. 4 is a cross-sectional view of a vein with an integral one-way valve of the suture of the present invention situated therein; and

FIG. 5 is a cross-sectional view of the vein of FIG. 4 showing further aspects of an intraluminal device of the suture of FIG. 1 situated therein.

### DETAILED DESCRIPTION OF THE PREFERRED EMBODIMENTS

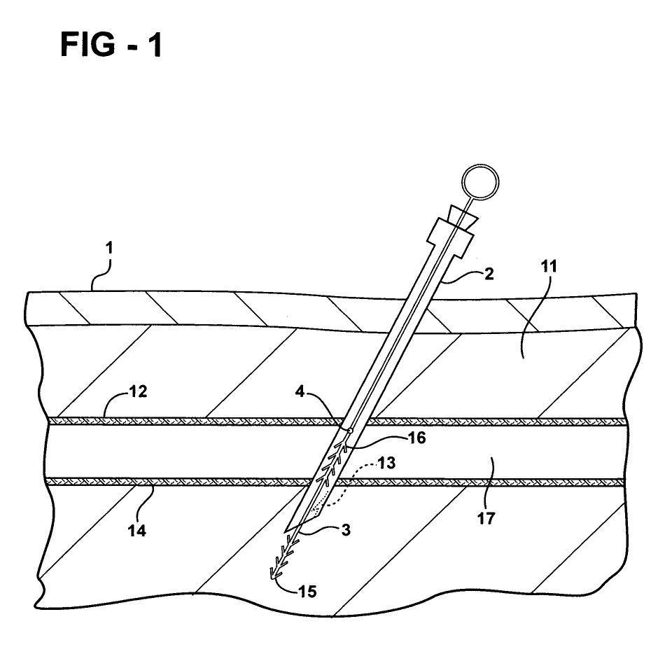

FIG. 1 is a cross-sectional perspective of a vein 12 and skin surface 1 showing a needle 2 penetrating the skin 1 and subcutaneous tissue 11 before penetrating the vein and placing tie distal barbs 15 of a bidirectional suture 3 on tie distal side of the vein. Flaring 6 of the proximal tip of the insertion device 2 is shown and provides an easier means to insert a bidirectional barbed suture into an insertion device. Alternatively, a needle-like design would also work, as would a straight tubular design. It should be noted that “barbed” in this specification refers to extensions from the sutures that allow movement in only one direction and can represent either cuts placed in the suture, integral structural elements manufactured in the suture, or elements added to the suture. “Bidirectional sutures” refer to extensions from the suture that prevent movement in either direction along the longitudinal axis, with barbs at one end preventing movement in tissue opposite the direction of movement prevented by barbs at the opposite end. Insertion device 2 shows an optimal flared proximal end 6 that allows easier insertion of the bidirectional suture 3.

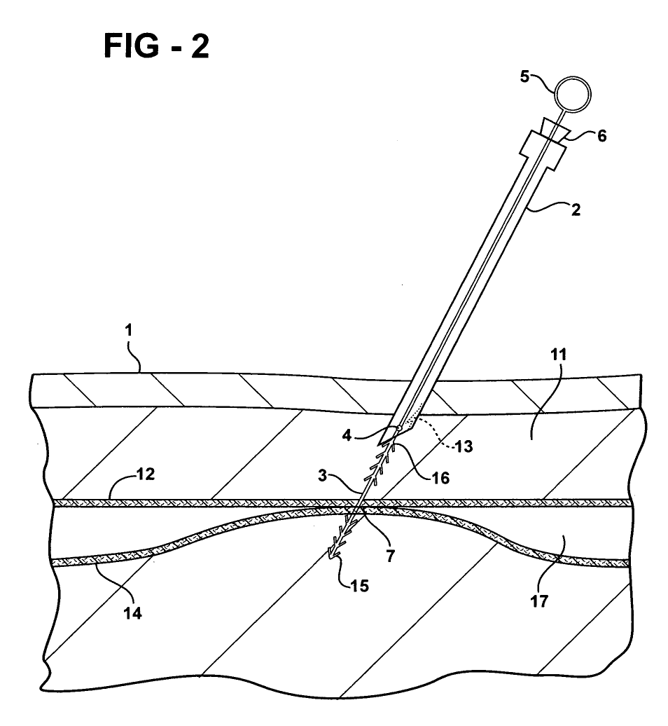

FIG. 2 is a cross-sectional perspective of a vein and overlying tissue and skin demonstrating the withdrawal of the insertion device 2 and simultaneous application of slight pressure to the pulling device 5 to pull the opposing vein walls 12 and 14 together. The pulling device 5 can take many forms, including the ring shape as shown, and is present to assist the surgeon in grasping the suture and applying force.

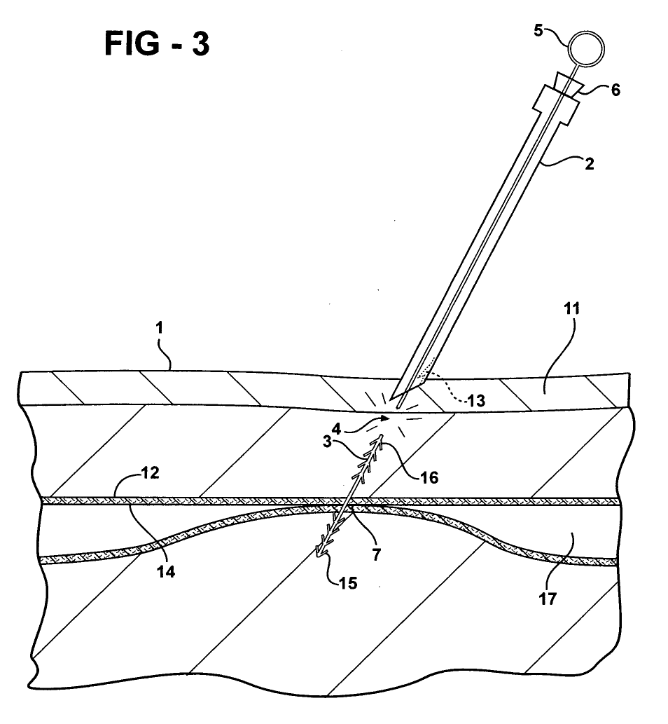

FIG. 3 is a cross-sectional perspective of a vein and overlying tissue and skin showing near complete withdrawal of the insertion device 2 after more tension has been applied to the pulling device 5 causing complete severance of the suture 3 at a predetermined breaking point 4 just proximal to the most proximal barb 16. Breaking point 4 can be manufactured into the suture by partially cutting the suture, making it thinner or of weaker materials, or by placing a device on or in the insertion device that causes severance of the suture material. All of these methods allow for enough tension to be placed on the bidirectional suture 3 to oppose vein walls 12 and 14 before the suture breaks at the predetermined breaking point 4.

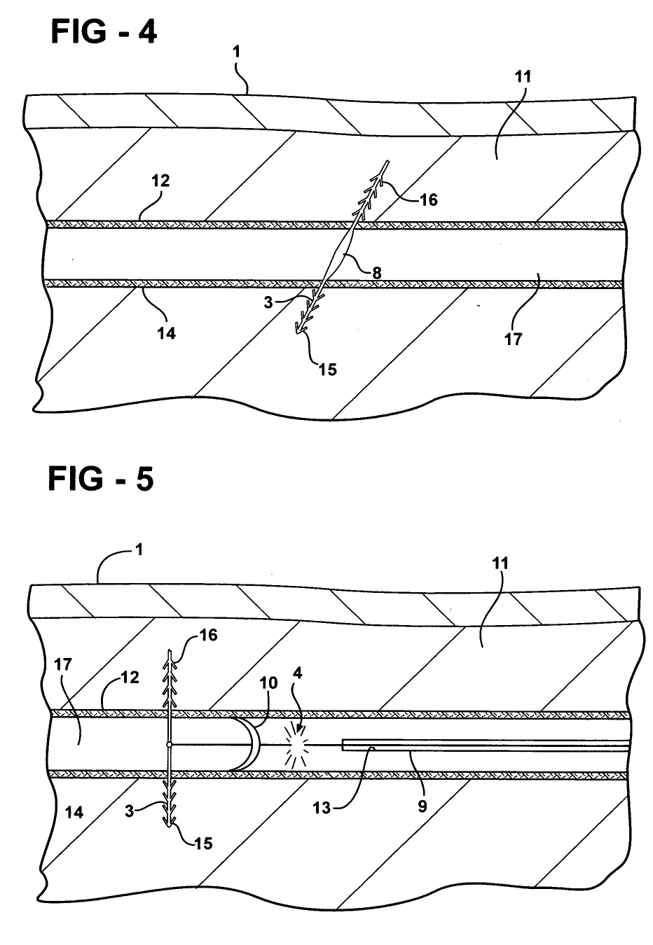

FIG. 4 is a cross-sectional perspective of a vein and overlying tissue and skin showing a bidirectional suture 3 with integral one-way valve 8 placed in the lumen 17 of the vein between walls 12 and 14.

FIG. 5 is a cross-sectional perspective of a vein and overlying tissue and skin showing the anchoring of an intraluminal device 10 that was placed by an intraluminal insertion device 9.

In operation, the insertion device 2, preloaded with the barbed suture 3, is introduced through the skin and subcutaneous tissues passing through the vein walls 12 and 14. The insertion device distal tip is then partially withdrawn into the vein exposing the distal barbs 15 in a position distal to the vein. Pulling on the pulling device results in direct vein wall opposition 7. Withdrawal of the insertion device tip into the subcutaneous tissue above the vein then exposes proximal barbs 16. Additional pulling pressure on the pulling device 5 causes complete severance of a predetermined breaking point 4 in suture 3. The insertion device tip is then completely withdrawn outside of the skin leaving the bidirectional suture in a position that is holding the vein walls 12 and 14 in an approximated position 7.

Several effects are noted when the barbed suture is placed in this manner: (1) blood flow is stopped within the vein; (2) pooling of blood is reduced in that limb; (3) the sequelae of venous insufficiency is reduced or averted; (4) existing ulcerations may heal; (5) symptoms of venous insufficiency are reduced; and (6) venous pressures are reduced in that limb resulting in less potential for associated vein malfunction.

In a similar manner, a bidirectional suture with an integral one-way valve (8, FIG. 4) could be placed within the lumen 17 of an incompetent vein. Likewise, a one-way valve could be placed by an intraluminal device 9 and secured by a bidirectional suture (FIG. 5). The effects of either of these methods would be similar to the effects 1-6 listed above.

Although the description above contains many specifications, they should not be construed as limiting the scope of this invention, but merely providing illustrations of some of the presently preferred embodiments of this invention. For example, the bidirectional suture could be pushed from the inserting device by a plunger, spring-loaded device, or with pressurized fluid or gas. Thus, the scope of the invention should be determined by the appended claims and their legal equivalents rather than by the examples given.

1. A method of improving venous system blood flow to correct venous insufficiency using a bidirectional suture of the type including an elongated body, proximal end with predetermined breaking point and a pulling device and a plurality of barbs extending from tie peripheral of the body, the barbs on a first portion of the body between the first end of the suture and a first axial location on the body for permitting movement of the suture through the tissue in a direction of movement of the first end preventing movement of the suture relative to the tissue in a direction opposite of the direction of the movement in the first end and the barbs on a second portion of the body between the second end of the suture and a second axial location on the body which is less from the distance on the second end to the first axial location preventing movement of the suture through the tissue in a direction opposite of the direction of movement of the second end, and using an insertion device including a tubular element, the method comprising the steps of:
inserting the bidirectional suture into an insertion device;
pushing the insertion device with enclosed bidirectional suture into a patient's body through the vein to be treated;
pulling the insertion device tip partially into the vein lumen, leaving the suture with one set of barbs in the tissue opposite the vein;
applying a pulling pressure to the pulling device to oppose the vein walls;
further withdrawing the insertion device into the subcutaneous tissue to expose the opposing set of barbs proximal to the vein;
applying a final pulling pressure on the pulling device to separate the proximal suture at a predetermined breaking point and leave the bidirectional suture in place holding the vein walls in an approximated or opposed position; and
completely withdrawing the insertion device.

2. A method of improving venous system blood flow to correct venous insufficiency, as recited in claim 1, wherein the bidirectional suture has an integral one-way valve that is left in the lumen of the vein and the vein walls are not held in an approximated position.

3. A method of improving vein blood flow to correct venous insufficiency, as recited in claim 1, wherein a bidirectional suture is placed through a vein to secure a one-way valve of other device placed by an intraluminal instrument.

4. A method of improving venous system blood flow to correct venous insufficiency, as recited in claim 1, wherein:
a rigid or semi-rigid bidirectional suture with a pointed distal tip is utilized either through an insertion device; or
the pointed distal tip of the bidirectional suture is utilized to penetrate the person's body and vein and a tubular device is used to cover the proximal barbs of the bidirectional suture.

5. A method of improving venous system blood flow to correct venous insufficiency, as recited in claim 1, wherein the bidirectional suture utilizes a pulling device.

6. A method of improving venous system blood flow to correct venous insufficiency, as recited in claim 1, wherein the bidirectional suture has a predetermined breaking point to aid in placement.

7. A method of improving venous system blood flow to correct venous insufficiency, as recited in claim 1, wherein the insertion device has a flared proximal end to aid in loading of the bidirectional suture.

8. A method of improving venous system blood flow to correct venous insufficiency, as recited in claim 3, wherein a sclerosant is utilized in the proximal, superficial venous system, after bidirectional suture placement to assure complete destruction of the closed vein.

9. A method of improving venous system blood flow to correct venous insufficiency, as recited in claim 1, wherein a duplex imaging device or fluoroscopy is utilized to guide placement of the bidirectional suture.

10. A method for joining or holding closed body tissue in wounds, attaching devices to tissue, attaching dissimilar body tissues, and altering the position of body tissue using a bidirectional suture with a predetermined breaking point.

11. A method for holding closed body tissue in wounds, attaching devices to tissue, attaching dissimilar body tissues, and altering the position of body tissue using a bidirectional suture and predetermined breaking point, as recited in claim 10, wherein these functions are performed remotely through percutaneous access.

12. A method for holding closed body tissue in wounds, attaching devices to tissue, attaching dissimilar body tissues, and altering the position of body tissue using a bidirectional suture and predetermined breaking point, as recited in claim 10, wherein these functions are performed through an endoscopic device.

13. A method for holding closed body tissue in wounds, attaching devices to tissue, attaching dissimilar body tissues, and altering the position of body tissue using a bidirectional suture and predetermined breaking point, as recited in claim 10, wherein these functions are performed utilizing an ultrasound device or fluoroscopy to guide placement of the bidirectional suture.

14. A method of improving venous system blood flow to correct venous insufficiency, as recited in claim 2, wherein a unidirectional suture with barbs allowing movement in only one direction and with integral one-way valve at the distal end, said distal end left in the lumen of the vein and vein walls are not directly opposed at completion of insertion.

15. A method of improving venous system blood flow to correct venous insufficiency, wherein an endoscopic device is utilized to ligate incompetent veins, wherein said device contains an ultrasound device to detect abnormal blood flow, thereby allowing the operator to distinguish between competent and incompetent veins before ligation is performed.

16. A method for holding closed body tissue in wounds, attaching devices to tissue, attaching dissimilar body tissues, and altering the position of body tissue using a suture with a plurality of barbs, said individual barbs designed to prevent movement in both directions along the longitudinal axis of the suture.

17. A method of improving venous system blood flow to correct venous insufficiency, wherein a length of expandable substance is placed within the lumen of an incompetent vein, said expandable substance, when exposed to blood or other fluid, increases its own dimensions, primarily along its width, such as to cause complete obstruction of blood flow, said expandable substance and the vein within which it is encompassed being absorbed over time by the body, resulting in elimination of the incompetent vein segment.
